(19) United States
(12) Patent Application Publication
(10) Pub. No.: US 2025/0375487 A1
(43) Pub. Date: Dec. 11, 2025

(54) USE OF A MICROBIOTA-MODULATING AGENT FOR ENHANCING BONE DEVELOPMENT AND/OR BONE STRENGTH

(71) Applicant: SOCIETE DES PRODUITS NESTLE S.A., Vevey (CH)

(72) Inventors: MARIE NOELLE HORCAJADA, Echenevex (FR); NICOLAS BONNET, Beaumont (FR)

(21) Appl. No.: 18/877,530

(22) PCT Filed: Jun. 19, 2023
(86) PCT No.: PCT/EP2023/066415

§ 371 Date: Dec. 20, 2024

(30) Foreign Application Priority Data
Jun. 22, 2022 (EP) 22180317.4

(51) Int. Cl. A61K 35/742; A61K 31/702; A61P 19/00

(52) U.S. Cl. CPC A61K 35/742; A61K 31/702; A61P 19/00

(57) ABSTRACT

The invention relates to the use of a microbiota-modulating agent for enhancing bone development and/or bone strength. wherein the microbiota-modulating agent modulates the abundance of one or more bacterial taxa selected from Intestinimonas butyriciproducens, Turicibacter sanguinis, Paraprevotella clara, and Acidaminococcaceae in the gut of the subject.

### TECHNICAL FIELD

The present invention relates to the use of a microbiota-modulating agent for enhancing bone development and/or bone strength in a subject.

### BACKGROUND OF THE INVENTION

Bone is a dynamic tissue which undergoes remodelling, a life-long process consisting of resorption (the breaking down of old bone) and ossification, that is key to shaping the skeleton and to repairing bone fractures. Ossification, or osteogenesis, is the process of bone formation by osteoblasts.

Bone growth during infancy is a key parameter with respect to bone strength during human life. Ossification begins approximately six weeks after fertilization in an embryo. Before this time, the embryonic skeleton consists entirely of fibrous membranes and hyaline cartilage.

Bone growth continues until approximately age 25. Bones can grow in thickness throughout life, but after age 25, ossification functions primarily in bone remodelling and repair.

Bone strength is the joint result of the action of several parameters which are involved in bone growth. In this respect, not only factors such as age, gender, location in the body, mineral content, disease etc. have an impact on bone strength, but also the trabecular architecture (form and orientation of trabeculae) and the cortical microarchitecture (in particular, as regards the repartition of porosity) play a significant role in bone health and strength.

Bone is composed of cortical (or compact) bone and trabecular (or spongy) bone. Cortical bone accounts for approximately 80% of the mass of bone of the human body and has a lower surface area than trabecular bone due to its lower porosity. Trabecular bone is located at the end of long bones and accounts for approximately 20% of the total mass of the skeleton.

Breast feeding is considered as the ideal source of nutrition and is the preferred choice for feeding infants up to at least 6 months of age. Consequently, human milk (HM) has long been considered as the model for the design of infant formulas (IF). Even though many improvements in the nutrient composition of IF have been made during the last decades, there are still important differences in composition as well as in functional benefits conveyed by HM. As a consequence, regarding bone metabolism, a lower bone mineral density can, for example, be observed in formula fed infants compared to breastfed ones.

Therefore, there is a need for new approaches to enhance bone development and/or bone strength, particularly in formula fed infants.

### SUMMARY OF THE INVENTION

It has now been surprisingly found that bone development and/or bone strength in a subject is associated with the abundance of Intestinimonas butyriciproducens, Turicibacter sanguinis, Paraprevotella clara, and Acidaminococcaceae in the gut of the subject. In particular, it was surprisingly found that the abundances of Intestinimonas butyriciproducens and Turicibacter sanguinis are positively associated with bone outcomes and that the abundances of Paraprevotella clara and Acidaminococcaceae are negatively associated with bone outcomes.

In one aspect, the present invention provides a microbiota-modulating agent for modulating the abundance of one or more bacterial taxa selected from Intestinimonas butyriciproducens, Turicibacter sanguinis, Paraprevotella clara, and Acidaminococcaceae in the gut of a subject.

The microbiota-modulating agent may increase the abundance of Intestinimonas butyriciproducens and/or Turicibacter sanguinis in the gut of the subject and/or the microbiota-modulating agent may decrease the abundance of Paraprevotella clara and/or Acidaminococcaceae in the gut of the subject. The microbiota-modulating agent may increase the abundance of Intestinimonas butyriciproducens and Turicibacter sanguinis in the gut of the subject and/or the microbiota-modulating agent may decrease the abundance of Paraprevotella clara and Acidaminococcaceae in the gut of the subject.

In some embodiments, the microbiota-modulating agent is Intestinimonas butyriciproducensand/or Turicibacter sanguinis. In some embodiments, the microbiota-modulating agent is a probiotic comprising or consisting of Intestinimonas butyriciproducens and/or Turicibacter sanguinis. The Intestinimonas butyriciproducens and/or Turicibacter sanguinis may be administered in a dose of 1E6 to 1E10 cfu/day.

In some embodiments, the microbiota-modulating agent is a mixture of oligosaccharides comprising at least one sialylated oligosaccharide, at least one fucosylated oligosaccharide, and/or at least one N-acetylated oligosaccharide. In some embodiments, the microbiota-modulating agent is a prebiotic comprising or consisting of at least one sialylated oligosaccharide, at least one fucosylated oligosaccharide, and/or at least one N-acetylated oligosaccharide.

In some embodiments, the microbiota-modulating agent is a combination comprising a mixture of oligosaccharides and Intestinimonas butyriciproducens and/or Turicibacter sanguinis, wherein said mixture of oligosaccharides comprises at least one sialylated oligosaccharide, at least one fucosylated oligosaccharide, and/or at least one N-acetylated oligosaccharide. The mixture of oligosaccharides and Intestinimonas butyriciproducens and/or Turicibacter sanguinis may be administered separately, simultaneously or sequentially. In some embodiments, the mixture of oligosaccharides and Intestinimonas butyriciproducens and/or Turicibacter sanguinis is administered simultaneously. In some embodiments, the microbiota-modulating agent is a synbiotic comprising or consisting of: (i) Intestinimonas butyriciproducens and/or Turicibacter sanguinis; and/or (ii) a mixture of oligosaccharides comprising at least one sialylated oligosaccharide, at least one fucosylated oligosaccharide, and/or at least one N-acetylated oligosaccharide.

In some embodiments, the mixture of oligosaccharides comprises at least one sialylated oligosaccharide. The at least one sialylated oligosaccharide may be selected from the group consisting of 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof. In some embodiments, the at least one sialylated oligosaccharide is selected from 3′-sialyllactose (3′-SL), 6′-sialyllactose (6′-SL) and combinations thereof. In some embodiments, the mixture of oligosaccharides comprises or consists of 3′-sialyllactose (3′-SL) and 6′-sialyllactose (6′-SL).

In preferred embodiments, the mixture of oligosaccharides comprises at least one fucosylated oligosaccharide. The at least one fucosylated oligosaccharide may be selected from the group consisting of 2′-fucosyllactose (2′FL), 3-fucosyllactose (3FL), difucosyllactose (diFL) (also known as 2′,3-difucosyllactose (LDFT)), lacto-N-fucopentaose-I (LNFP-I), lacto-N-fucopentaose-II (LNFP-II), lacto-N-fucopentaose-III (LNFP-III), lacto-N-fucopentaose-V (LNFP-V), lacto-neofucopentaose V (LNnFP-V), lacto-N-difucosylhexaose-I (LNDFH-1), lacto-N-neodifucosylhexaose (LNnDFH), monofucosyllacto-n-hexaose-III (MFNLH-III), difucosyllacto-N-hexaose-a (DFLNHa) and combinations thereof. In some embodiments, the at least one fucosylated oligosaccharide is selected from the group consisting of 2′-fucosyllactose (2′FL), difucosyllactose (diFL), and combinations thereof. In some embodiments, the mixture of oligosaccharides comprises or consists of 2′-fucosyllactose (2′FL) and difucosyllactose (diFL).

In preferred embodiments, the mixture of oligosaccharides comprises at least one N-acetylated oligosaccharide. The at least one N-acetylated oligosaccharide may be selected from the group consisting of N-acetyl-glucosamine, N-acetyl-galactosamines, lacto-N-tetraose (LNT), lacto-N-neotetraose (LNnT), and combinations thereof. In some embodiments, the at least one N-acetylated oligosaccharide is selected from lacto-N-tetraose (LNT), lacto-N-neotetraose (LNnT) and combinations thereof. In some embodiments, the mixture of oligosaccharides comprises or consists of lacto-N-tetraose (LNT) and lacto-N-neotetraose (LNnT).

In preferred embodiments, the mixture of oligosaccharides comprises or consists of 2′-fucosyllactose (2′FL), difucosyllactose (diFL), lacto-N-tetraose (LNT) and lacto-N-neotetraose (LNnT). In some embodiments, the mixture of oligosaccharides comprises or consists of 3′-sialyllactose (3′-SL), 6′-sialyllactose (6′-SL), 2′-fucosyllactose (2′FL), difucosyllactose (diFL), lacto-N-tetraose (LNT) and lacto-N-neotetraose (LNnT).

In some embodiments, the mixture of oligosaccharides comprises:
10 to 35 wt %, preferably 10 to 30 wt %, more preferably 10 to 25 wt %, with respect to the total weight of the oligosaccharide mixture, of at least one sialylated oligosaccharide;
30 to 80 wt %, preferably 40 to 80 wt %, more preferably 50 to 70 wt %, with respect to the total weight of the oligosaccharide mixture, of at least one fucosylated oligosaccharide; and/or
10 to 35 wt %, preferably 15 to 30 wt %, more preferably 15 to 20 wt %, with respect to the total weight of the oligosaccharide mixture, of at least one N-acetylated oligosaccharide.

The microbiota-modulating agent may be provided in any suitable form. The microbiota-modulating agent may be in the form of a nutritional composition, a medical food product for clinical nutrition, or a supplement.

The microbiota-modulating agent may be in the form of a nutritional composition, more preferably a synthetic nutritional composition.

In preferred embodiments, the microbiota-modulating agent is in the form of an infant formula. In this case, said infant formula can be a preterm infant formula, a human milk fortifier, a starter infant formula, a follow-on formula, a baby-food formula, an infant cereal formula, or a growing-up milk.

The microbiota-modulating agent may be in the form of a supplement. A supplement can be for e.g. a preterm infant or a child or an adult.

In preferred embodiments, the microbiota-modulating agent is in a form for preterm feeding such as a preterm infant formula, a human milk fortifier, or a supplement. The microbiota-modulating agent can also be in a form for children or adults such as yoghurt or medical food, as well as pet's food, especially young pets.

The microbiota-modulating agent can be for use before and/or during and/or after a weaning period. The microbiota-modulating agent can be used during hospital stay and/or after hospital discharge.

In some embodiments, the microbiota-modulating agent is in the form of a composition (e.g. an infant formula) comprising:
1 0.01 to 2 wt %, preferably 0.05 to 1.5 wt %, most preferably 0.07% to 1 wt % of at least one sialylated oligosaccharide;
0.05 to 3 wt %, preferably 0.1 to 2 wt %, most preferably 0.2 to 1.5 wt % of at least one fucosylated oligosaccharide; and/or
0.01 to 1 wt %, preferably 0.03 to 0.6 wt %, most preferably 0.05 to 0.5 wt % of at least one N-acetylated oligosaccharide.

The wt % may refer to the total weight of the composition in powder form.

In one aspect, the present invention provides a microbiota-modulating agent for use in enhancing bone development and/or bone strength in a subject, wherein the microbiota-modulating agent modulates the abundance of one or more bacterial taxa selected from Intestinimonas butyriciproducens, Turicibacter sanguinis, Paraprevotella clara, and Acidaminococcaceae in the gut of the subject.

In one aspect, the present invention provides a method for enhancing bone development and/or bone strength in a subject, said method comprising administering a therapeutically effective amount of a microbiota-modulating agent to a subject in need thereof, wherein the microbiota-modulating agent modulates the abundance of one or more bacterial taxa selected from Intestinimonas butyriciproducens, Turicibacter sanguinis, Paraprevotella clara, and Acidaminococcaceae in the gut of the subject.

The microbiota-modulating agent may be any microbiota-modulating agent for modulating the abundance of one or more bacterial taxa selected from Intestinimonas butyriciproducens, Turicibacter sanguinis, Paraprevotella clara, and Acidaminococcaceae in the gut of a subject described herein.

In some embodiments, the subject is a human or an animal. In preferred embodiments, the subject is a human. In some embodiments, the subject is a juvenile, an adolescent, a child, a toddler, or an infant. In preferred embodiments, the subject is an infant. In other embodiments, the subject is an adult.

The enhancement of bone development and/or bone strength may comprise at least one of the following physiological processes: bone catch-up growth, bone mass acquisition, optimization of peak bone mass, promotion of bone formation, promotion of bone anabolism, increase of bone mineral density and micro-architecture, modulation of bone biomechanical properties, modulation the ratio of bone formation and/or bone resorption, assist bone regeneration during fracture healing, regulation of bone resorption process.

The microbiota-modulating agent may increase one or more bone parameter selected from: bone mineral density (BMD), trabecular bone volume fraction (BV/TV), cortical bone volume (Ct.BV), and bone ultimate force (FMax).

### BRIEF DESCRIPTION OF THE FIGURES

FIG. 1 shows that Turicibacter sanguinis is positively associated with bone strength (biomechanics) and its relative abundance is increased by HMO blends. (A) shows that the relative abundance of Turicibacter sanguinis is positively correlated with bone strength. (B) shows that HMO blends A, B, and C each increased the relative abundance of Turicibacter sanguinis compared to lactose.

FIG. 2 shows that Paraprevotella clara is negatively associated with bone microarchitecture (connectivity) and its relative abundance is decreased by HMO blends. (A) shows that the relative abundance of Paraprevotella clara is negatively correlated with trabecular structure. (B) shows that HMO blends A, B, and C each decreased the relative abundance of Paraprevotella clara compared to lactose.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

Any reference to prior art documents in this specification is not to be considered an admission that such prior art is widely known or forms part of the common general knowledge in the field. All publications mentioned in the specification are herein incorporated by reference.

As used in this specification, the words “comprises”, “comprising”, and similar words, are not to be interpreted in an exclusive or exhaustive sense. In other words, they are intended to mean “including, but not limited to”. The terms “comprises”, “comprising”, and similar words also include the term “consisting of”.

The practice of the present invention will employ, unless otherwise indicated, conventional techniques which are within the capabilities of one of ordinary skill in the art. Such techniques are explained in the literature. Unless defined otherwise, all technical and scientific terms used herein have the same meaning as commonly understood by one of ordinary skill in the art.

Numeric ranges are inclusive of the numbers defining the range and all percentages disclosed herein are on a w/w basis, unless stated otherwise.

### Microbiota-Modulating Agent

In one aspect, the present invention provides a microbiota-modulating agent for modulating the abundance of one or more bacterial taxa selected from Intestinimonas butyriciproducens, Turicibacter sanguinis, Paraprevotella clara, and Acidaminococcaceae in the gut of a subject.

As used herein, the term “microbiota-modulating agent” or “gut microbiota-modulating agent” may refer to any agent which modulates the gut microbiota of a subject. Examples of such agents include probiotics, prebiotics, postbiotics, antibiotics, and fecal microbiota transplants. Suitably, the microbiota-modulating agent is in the form of a probiotic, prebiotic, and/or synbiotic. Suitably, the microbiota-modulating agent of the present invention comprises or consists of a probiotic, prebiotic, and/or synbiotic.

In some embodiments, the microbiota-modulating agent of the present invention is in the form of a probiotic. In some embodiments, the microbiota-modulating agent of the present invention comprises or consists of a probiotic. As used herein, the term “probiotic” may refer to a component that contains a sufficient number of viable microorganisms to alter the gut microbiota of the subject (see e.g. Hill, C., et al., 2014. Nature reviews Gastroenterology & hepatology, 11(8), p. 506).

In some embodiments, the microbiota-modulating agent of the present invention is in the form of a prebiotic. In some embodiments, the microbiota-modulating agent of the present invention comprises or consists of a prebiotic. As used herein, the term “prebiotic” may refer to a non-digestible component that benefits the subject by selectively stimulating the favourable growth and/or activity of one or more bacterial taxa (see e.g. Gibson, G. R., et al., 2017. Exemplary prebiotics include oligosaccharides, such as human milk oligosaccharides.

In some embodiments, the microbiota-modulating agent of the present invention is in the form of a synbiotic. In some embodiments, the microbiota-modulating agent of the present invention comprises or consists of a synbiotic. As used herein, the term “synbiotic” may refer to a component that contains both probiotics and prebiotics (see e.g. Swanson, K. S., et al., 2020. Nature Reviews Gastroenterology & Hepatology, 17(11), pp. 687-701).

### Intestinimonas butyriciproducens and Turicibacter Sanguinis

The microbiota-modulating agent may increase the abundance of Intestinimonas butyriciproducens and/or Turicibacter sanguinis in the gut of the subject.

In some embodiments, the microbiota-modulating agent is Intestinimonas butyriciproducens and/or Turicibacter sanguinis. In some embodiments, the microbiota-modulating agent comprises or consists of Intestinimonas butyriciproducens and/or Turicibacter sanguinis.

In some embodiments, the microbiota-modulating agent increase the abundance of Intestinimonas butyriciproducens in the gut of the subject. In some embodiments, the microbiota-modulating agent is Intestinimonas butyriciproducens. In some embodiments, the microbiota-modulating agent comprises or consists of Intestinimonas butyriciproducens. Intestinimonas butyriciproducens is a butyrate-producing species commonly found in the intestine (see e.g. Bui, T. P. N., et al., 2016. Environmental microbiology reports, 8(6), pp. 1024-1037). Any suitable strain of Intestinimonas butyriciproducens may be used. Intestinimonas butyriciproducens may be routinely cultured. For example, I. butyriciproducens strain AF211 may be cultivated in anaerobic Reinforced Clostridium Medium at 37° C. under a gas phase of N2/CO2 (80:20, v/v) (see Bui, T. P. N., et al., 2016. Environmental microbiology reports, 8 (6), pp. 1024-1037).

In some embodiments, the microbiota-modulating agent increase the abundance of Turicibacter sanguinis in the gut of the subject. In some embodiments, the microbiota-modulating agent is Turicibacter sanguinis. In some embodiments, the microbiota-modulating agent comprises or consists of Turicibacter sanguinis. Turicibacter sanguinis is commonly found in intestine and is capable of interaction with host-derived compounds (see e.g. Cuiv, P. O., et al., 2011. Journal of bacteriology, 193(5), pp. 1288-1289). Any suitable strain of Turicibacter sanguinis may be used. Turicibacter sanguinis may be routinely cultured. For example, T. sanguinis strain MOL361^{T} may be cultivated on different standard media under anaerobic and aerobic conditions (see Bosshard, P. P., et al., 2002. International journal of systematic and evolutionary microbiology, 52(4), pp. 1263-1266).

The microbiota-modulating agent may provide any suitable amount of Intestinimonas butyriciproducens and/or Turicibacter sanguinis.

Suitably, the Intestinimonas butyriciproducens and/or Turicibacter sanguinis is administered in a dose of at least 1E5 cfu/day, at least 1E6 cfu/day, at least 1E7 cfu/day, at least 1E8 cfu/day, at least 1E9 cfu/day, or at least 1E10 cfu/day. In some embodiments, the Intestinimonas butyriciproducens and/or Turicibacter sanguinis is administered in a dose of 1E5 to 1E10 cfu/day or 1E6 to 1E10 cfu/day.

Suitably, the microbiota-modulating agent comprises or consists of Intestinimonas butyriciproducens and/or Turicibacter sanguinis in an amount of at least 1E4 cfu/ml, at least 1E5 cfu/ml, at least 1E6 cfu/ml, at least 1E7 cfu/ml, at least 1E8 cfu/ml, or at least 1E9 cfu/ml cfu/ml. In some embodiments, the microbiota-modulating agent comprises or consists of Intestinimonas butyriciproducens and/or Turicibacter sanguinis in an amount of 1E4 to 1E9cfu/ml or 1E5 to 1E9 cfu/ml.

Suitably, the microbiota-modulating agent comprises Intestinimonas butyriciproducens and/or Turicibacter sanguinis in an amount of at least 1E4 cfu/g, at least 1E5 cfu/g, at least 1E6 cfu/g, at least 1E7 cfu/g, at least 1E8 cfu/g, or at least 1E9 cfu/g. In some embodiments, the microbiota-modulating agent comprises or consists of Intestinimonas butyriciproducens and/or Turicibacter sanguinis in an amount of 1E4 to 1E9 cfu/g or 1E5 to 1E9 cfu/g.

### Paraprevotella clara and Acidaminococcaceae

The microbiota-modulating agent may decrease the abundance of Paraprevotella clara and/or Acidaminococcaceae in the gut of the subject.

In some embodiments, the microbiota-modulating agent decreases the abundance of Paraprevotella clara in the gut of the subject. Paraprevotella clara is a member of the Prevotellaceae family that has been isolated from human faeces (see e.g. Morotomi, M., et al., 2009. International Journal of Systematic and Evolutionary Microbiology, 59(8), pp. 1895-1900).

In some embodiments, the microbiota-modulating agent decreases the abundance of Acidaminococcaceae in the gut of the subject. Acidaminococcaceae is a genus of bacteria, whose members can use amino acids as the sole energy source for growth and that that has been isolated from human gut samples (see e.g. Ricaboni, D., et al., 2017. New Microbes and New Infections, 15, pp. 46-48). Species of Acidaminococcus include Acidaminococcus intestini and Acidaminococcus fermentans.

### Oligosaccharide Mixture

In some embodiments, the microbiota-modulating agent is a mixture of oligosaccharides. In some embodiments, the microbiota-modulating agent comprises or consists of a mixture of oligosaccharides.

Oligosaccharides are one of the best know prebiotics. The oligosaccharides may be any suitable oligosaccharides for modulating the abundance of one or more bacterial taxa selected from Intestinimonas butyriciproducens, Turicibacter sanguinis, Paraprevotella clara, and Acidaminococcaceae in the gut of a subject.

The oligosaccharides may be human milk oligosaccharides (HMOs). Many different kinds of HMOs are found in the human milk and are typically based on a combination of glucose, galactose, sialic acid (N-acetylneuraminic acid), fucose and/or N-acetylglucosamine with many and varied linkages between them. Almost all HMOs have a lactose moiety at their reducing end while sialic acid and/or fucose (when present) occupy terminal positions at the non-reducing ends. HMOs can be acidic (e.g. charged sialic acid containing oligosaccharides) or neutral (e.g. fucosylated oligosaccharides).

Suitably, the mixture of oligosaccharides may comprise at least one sialylated oligosaccharide, at least one fucosylated oligosaccharide, and/or at least one N-acetylated oligosaccharide. In some embodiments, the mixture of oligosaccharides comprises or consists of at least one sialylated oligosaccharide, at least one fucosylated oligosaccharide, and at least one N-acetylated oligosaccharide.

In some embodiments, the mixture of oligosaccharides comprises 10 to 35 wt %, preferably 10 to 30 wt %, more preferably 10 to 25 wt %, with respect to the total weight of the oligosaccharide mixture, of at least one sialylated oligosaccharide.

In some embodiments, the mixture of oligosaccharides comprises 30 to 80 wt %, preferably 40 to 80 wt %, more preferably 50 to 70 wt %, with respect to the total weight of the oligosaccharide mixture, of at least one fucosylated oligosaccharide.

In some embodiments, the mixture of oligosaccharides comprises 10 to 35 wt %, preferably 15 to 30 wt %, more preferably 15 to 20 wt %, with respect to the total weight of the oligosaccharide mixture, of at least one N-acetylated oligosaccharide.

In some embodiments, the mixture of oligosaccharides comprises or consists of:
30 to 80 wt %, preferably 40 to 80 wt %, more preferably 50 to 70 wt %, with respect to the total weight of the oligosaccharide mixture, of at least one fucosylated oligosaccharide; and
10 to 35 wt %, preferably 15 to 30 wt %, more preferably 15 to 20 wt %, with respect to the total weight of the oligosaccharide mixture, of at least one N-acetylated oligosaccharide.

In some embodiments, the mixture of oligosaccharides comprises or consists of:
10 to 35 wt %, preferably 10 to 30 wt %, more preferably 10 to 25 wt %, with respect to the total weight of the oligosaccharide mixture, of at least one sialylated oligosaccharide;
30 to 80 wt %, preferably 40 to 80 wt %, more preferably 50 to 70 wt %, with respect to the total weight of the oligosaccharide mixture, of at least one fucosylated oligosaccharide; and
10 to 35 wt %, preferably 15 to 30 wt %, more preferably 15 to 20 wt %, with respect to the total weight of the oligosaccharide mixture, of at least one N-acetylated oligosaccharide.

The oligosaccharides may be obtained by any suitable method. Suitable methods for synthesising oligosaccharides will be well known to those of skill in the art. For example, processes have been developed for producing oligosaccharides by microbial fermentations, enzymatic processes, chemical syntheses, or combinations of these technologies (see e.g. Zeuner et al., 2019. Molecules, 24(11), p. 2033).

### Sialylated Oligosaccharides

In some embodiments, the microbiota-modulating agent is a mixture of oligosaccharides comprising or consisting of at least one sialylated oligosaccharide. In some embodiments, the microbiota-modulating agent comprises or consists of a mixture of oligosaccharides comprising or consisting of at least one sialylated oligosaccharide.

Non-limiting examples of sialylated oligosaccharides include: 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof.

In a preferred embodiment, the at least one sialylated oligosaccharide is selected from the group consisting of 3′-sialyllactose (3′-SL), 6′-sialyllactose (6′-SL) and combinations thereof.

As used herein the term “3′-sialyllactose”(3′-SL, 3-SL, 3′SL, or 3SL), refers to (6R)-5-Acetamido-3,5-dideoxy-6-[(1R,2R)-1,2,3-trihydroxypropyl]-β-L-threo-hex-2-ulopyranonosyl-(2->3)-β-D-galactopyranosyl-(1->4)-D-glucopyranose (IUPAC).

As used herein the term “6′-sialyllactose”(6′-SL, 6-SL, 6′SL, or 6SL) refers to (6R)-5-Acetamido-3,5-dideoxy-6-[(1R,2R)-1,2,3-trihydroxypropyl]-β-L-threo-hex-2-ulopyranonosyl-(2->6)-β-D-galactopyranosyl-(1->4)-D-glucopyranose (IUPAC).

The sialylated oligosaccharides may be obtained by any suitable method. For example, 3′-sialyllactose (3′-SL), and/or 6′-sialyllactose (6′-SL) may be isolated by chromatographic or filtration technology from a natural source such as animal milks. Alternatively, they may be produced by biotechnological means using specific sialyltransferases or sialidases, neuraminidases, either by an enzyme based fermentation technology (recombinant or natural enzymes), by chemical synthesis or by a microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any degree of polymerisation (DP), from DP=1 onwards. Alternatively, sialyllactoses may be produced by chemical synthesis from lactose and free N′-acetylneuraminic acid (sialic acid). Sialyllactoses are also commercially available for example from Kyowa Hakko Kogyo, Japan, or from GeneChem, Republic of Korea.

### Fucosylated Oligosaccharides

In some embodiments, the microbiota-modulating agent is a mixture of oligosaccharides comprising or consisting of at least one fucosylated oligosaccharide. In some embodiments, the microbiota-modulating agent comprises or consists of a mixture of oligosaccharides comprising or consisting of at least one fucosylated oligosaccharide.

Non-limiting example(s) of fucosylated oligosaccharide(s) include: 2′-fucosyllactose (2′FL), 3-fucosyllactose (3FL), difucosyllactose (diFL), lacto-N-fucopentaose, such as lacto-N-fucopentaose I (LNFP-I), lacto-N-fucopentaose II (LNFP-II), lacto-N-fucopentaose III (LNFP-III) or lacto-N-fucopentaose V (LNFP-V), lacto-N-fucohexaose, lacto-N-difucohexaose I, lacto-neofucopentaose V (LNnFP-V), lacto-N-difucosylhexaose-I (LNDFH-1), lacto-N-neodifucosylhexaose (LNnDFH), fucosyllacto-N-hexaose, fucosyllacto-N-neohexaose (such as fucosyllacto-N-neohexaose I, fucosyllacto-N-neohexaose II), monofucosyllacto-n-hexaose-III (MFNLH-III), difucosyllacto-N-hexaose I, difuco-lacto-N-neohexaose, difucosyllacto-N-neohexaose I, difucosyllacto-N-neohexaose II, difucosyllacto-N-hexaose-a (DFLNHa), fucosyl-para-Lacto-N-hexaose, tri-fuco-para-Lacto-N-hexaose I, and combinations thereof.

In a preferred embodiment, the at least one fucosylated oligosaccharide is selected from the group consisting of 2′-fucosyllactose (2′FL), difucosyllactose (diFL) and combinations thereof.

The fucosylated oligosaccharides may be obtained by any suitable method. For example, 2′FL may be produced by biotechnological means using specific fucosyltransferases and/or fucosidases either through the use of enzyme-based fermentation technology (recombinant or natural enzymes) or microbial fermentation technology. In the latter case, microbes may either express their natural enzymes and substrates or may be engineered to produce respective substrates and enzymes. Alternatively, 2′FL may be produced by chemical synthesis from lactose and free fucose. diFL may be synthesized by enzymatic, biotechnological and/or chemical processes.

### N-Acetylated Oligosaccharides

In some embodiments, the microbiota-modulating agent is a mixture of oligosaccharides comprising or consisting of at least one N-acetylated oligosaccharide. In some embodiments, the microbiota-modulating agent comprises or consists of a mixture of oligosaccharides comprising or consisting of at least one N-acetylated oligosaccharide.

Suitably, the at least one N-acetylated oligosaccharide is selected from the group consisting of N-acetyl-glucosamine, N-acetyl-galactosamines and combinations thereof. Non-limiting examples of N-acetylated oligosaccharide(s) include: LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

In a preferred embodiment, the at least one N-acetylated oligosaccharide is selected from the group consisting of lacto-N-tetraose (LNT), lacto-N-neotetraose (LNnT) and combinations thereof.

The N-acetylated oligosaccharides may be obtained by any suitable method. For example, LNnT may be synthesised chemically by enzymatic transfer of saccharide units from donor moieties to acceptor moieties using glycosyltransferases. Alternatively, LNnT may be prepared by chemical conversion of Keto-hexoses (e.g. fructose) either free or bound to an oligosaccharide (e.g. lactulose) into N-acetylhexosamine or an N-acetylhexosamine-containing oligosaccharide. LNT may be synthesized by enzymatic, biotechnological and/or chemical processes.

### Administration of Oligosaccharides

The oligosaccharides may be administered in any suitable from and in any suitable amount.

For example, for an infant formula or growing up milk, the skilled person may base the amount of an HMO e.g. 2′FL, diFL, LNT, LNnT, 3SL and/or 6SL on the amounts found in human breast milk produced for an infant or child of the same age, in particular by a nutritionally replete mother. Such amounts may fall within the following ranges in human breast milk: diFL: 100-500 mg/L, LNT: 50-300 mg/L, LNnT: 200-2000 mg/L, 2′FL: 500-3000 mg/L, 3′SL: 100-400 mg/L, 6′SL: 50-750 mg/L. However, they may be outside depending on for example bioavailability of said HMOs from infant formula in comparison to human breastmilk.

As a guide, for e.g. an infant formula or growing up milk, the fucosylated oligosaccharide(s) (e.g. 2′FL and/or diFL) may be present in a nutritional composition in a total amount of 0.1 to 4 g/L of the composition, for example in a total amount of 0.1 to 3.5 g/L of the composition for example 0.15 to 3 g/L, 0.2 to 2.5 g/L, 0.3 to 2 g/L, 0.4 to 2 g/L, 0.5 to 2 g/L of the composition (the concentration may refer to the concentration after the composition has been reconstituted e.g. with water). In a particular embodiment, a nutritional composition comprises from 200 to 1800 mg of total fucosylated oligosaccharide(s) per L of the nutritional composition.

As a guide, for e.g. an infant formula or growing up milk, the sialylated oligosaccharide(s) (e.g. 3′-sialyllactose (3′-SL) and/or 6′-sialyllactose (6′-SL) may be present in a nutritional composition in a concentration of from 50 mg to 750 mg/L for example from 50 mg to 500 mg/L for example from 100 mg to 300 mg per L, from 100 mg to 400 mg per L of the nutritional composition. In a particular embodiment, a nutritional composition comprises from 120 mg to 400 mg of total sialylated oligosaccharide(s) per L of the nutritional composition.

If a nutritional composition comprises 3′-Sialyllactose (3′-SL) and 6′-Sialyllactose (6′-SL), it may be particularly beneficial if said 3′-Sialyllactose (3′-SL) and 6′-Sialyllactose (6′-SL) are comprised in said nutritional composition in a weight ratio between 10:1 and 1:10, such as between 10:1 and 2:1, between 8:1 and 3:1, between 6:1 and 3:1, between 5:1 and 3:1, between 5:1 and 4:1, or else between 1:2 to 1.5:1.

As a guide, e.g. for an infant formula or growing up milk, the N-acetylated oligosaccharide(s) (e.g. LNT and/or LNnT) may be present in a nutritional composition in a total amount of 0.05 to 0.5 g/L of the composition, for example in a total amount of 0.1 to 0.5 g/L, 0.2 to 0.4 g/L, or 0.3 g/L of the composition, for example 0.35 g/L of the composition.

Suitably, a microbiota-modulating agent (e.g. in the form of a nutritional composition, such as an infant formula) may comprise:
at least one sialylated oligosaccharide in in a total amount of 0.05 to 0.75 g/L, 0.05 to 0.5 g/L, 0.1 to 0.3 g/L, or 0.1 to 0.4 mg/L;
at least one fucosylated oligosaccharide in a total amount of 0.1 to 4 g/L, 0.1 to 3.5 g/L, 0.15 to 3 g/L, 0.2 to 2.5 g/L, 0.3 to 2 g/L, 0.4 to 2 g/L, or 0.5 to 2 g/L; and/or
at least one N-acetylated oligosaccharide in a total amount of 0.05 to 0.5 g/L, 0.1 to 0.5 g/L, or 0.2 to 0.4 g/L.

Suitably, a microbiota-modulating agent (e.g. in the form of a nutritional composition, such as an infant formula) may comprise:
0.01 to 2 wt %, preferably 0.05 to 1.5 wt %, most preferably 0.07% to 1 wt % of at least one sialylated oligosaccharide;
0.05 to 3 wt %, preferably 0.1 to 2 wt %, most preferably 0.2 to 1.5 wt % of at least one fucosylated oligosaccharide; and/or
0.01 to 1 wt %, preferably 0.03 to 0.6 wt %, most preferably 0.05 to 0.5 wt % of at least one N-acetylated oligosaccharide.

The wt % may refer to the total dry weight of the composition (e.g. total weight of the composition in powder form).

### Compositions

Suitably, the microbiota-modulating agent may be in the form of a composition. The composition may comprises the microbiota-modulating agent in any therapeutically effective amount.

In some embodiments, the microbiota-modulating agent is in the form of a nutritional composition, an infant formula, a medical food product for clinical nutrition, or a supplement.

In some embodiments, the microbiota-modulating agent is in the form of a nutritional composition. As used herein, a “nutritional composition” may mean a composition which nourishes a subject. This nutritional composition is usually to be taken orally or intravenously, and it usually includes a lipid or fat source and a protein source.

In some embodiments, the microbiota-modulating agent is in the form of synthetic nutritional composition. As used herein, a “synthetic nutritional composition” may mean a mixture obtained by chemical and/or biological means, which can be chemically identical to the mixture naturally occurring in mammalian milks (i.e., the synthetic composition is not breast milk).

In some embodiments, the microbiota-modulating agent is in the form of a medical food product for clinical nutrition. As used herein, a “medical food product for clinical nutrition” may also be known as a “Food for Special Medical Purposes (FSMP)” and refer to specialised foods designed to help meet the nutritional or dietary needs of subjects living with a disease, disorder or medical condition who are temporarily or permanently unable to achieve an adequate nutritional intake from normal foods or through modification of the normal diet.

The composition can be any type of composition in which the microbiota-modulating agent can be incorporated, such as a composition in the form of a food or beverage product, an animal feed product, a nutritional supplement for human or animal, or a pharmaceutical composition. The composition may be in solid (e.g. powder), liquid or semi-liquid form. In some embodiments, the microbiota-modulating agent is in the form of a food composition, a pet food composition, a beverage, a nutritional formula, a nutritional supplement, or a nutraceutical.

Food and beverage products include all products intended to be consumed orally by human beings, for the purpose of providing nutrition and/or pleasure. It can for example be a nutritional composition, such as for infants and/or young children, or for individuals in need of a special nutrition due to an adverse health condition or for elderly people. Examples of food and beverage products include dairy products such as milk products or yogurts, soups, sauces, sweet and savoury snacks, powdered drinks and cereal products.

The composition can also be in the form of an animal food product or a nutritional supplement for animals. Preferably, the animal is a mammal. Examples of animals include primates (e.g., humans), cows, sheep, goats, horses, dogs, cats, rabbits, rats, mice, fish, birds and the like.

In preferred embodiments, the microbiota-modulating agent is in the form of an infant formula. In this case, said infant formula can be a preterm infant formula, a human milk fortifier, a starter infant formula, a follow-on formula, a baby-food formula, an infant cereal formula, or a growing-up milk.

The term “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

In some embodiments, the microbiota-modulating agent is in the form of a starter infant formula. Generally a “starter infant formula” is intended for infants from birth as breast-milk substitute.

In some embodiments, the microbiota-modulating agent is in the form of follow-on formula. A “follow-up formula” or “follow-on formula” may be given from the 6th month onwards. It may constitute the principal liquid element in the progressively diversified diet of this category of person.

In some embodiments, the microbiota-modulating agent is in the form of a preterm infant formula. The term “preterm infant formula” as used herein means an infant formula intended for a preterm infant.

In some embodiments, the microbiota-modulating agent is in the form of a milk fortifier. The term “milk fortifier” as used herein refers to liquid or solid nutritional compositions suitable for mixing with breast milk (which is human milk for a human milk fortifier) or infant formula. It is used to increase the calories, protein, minerals and vitamins in breast milk fed to preterm infants or infants with a low birth weight. The term “breast milk” is to be understood as the mother's milk or the colostrum of the mother or a donor's milk or the colostrum of a donor's milk.

In some embodiments, the microbiota-modulating agent is in the form of a baby-food formula. The term “baby food formula” as used herein means a foodstuff intended for particular nutritional use by infants or children such as young children, during the first years of life.

In some embodiments, the microbiota-modulating agent is in the form of a growing-up milk. The term “growing-up milk” (or GUM) as used herein refers to a milk formula product given from one year onwards. It is generally a diary-based beverage adapted for the specific nutritional needs of young children.

In some embodiments, the microbiota-modulating agent is in the form of an infant cereal composition. The term “infant cereal composition” as used herein refers to a foodstuff intended for particular nutritional use by infants or children such as young children, during the first years of life.

In another particular embodiment the microbiota-modulating agent is in the form of a fortifier. The fortifier can be a breast milk fortifier or a formula fortifier such as an infant formula fortifier. The fortifier is therefore a particularly advantageous embodiment when the infant or young child is born preterm.

In another particular embodiment the microbiota-modulating agent is in the form of a supplement. As used herein, a “supplement” or “dietary supplement” may be used to complement the nutrition of a subject (it is typically used as such but it might also be added to any kind of compositions intended to be ingested by the subject).

When the composition is a supplement, it can be provided in the form of unit doses. Supplements are typically present in the form of a liquid, a gel, a powder or a tablet or capsule. Powder supplements typically encompass supplements to be dissolved in water or to be sprinkled on food or in a beverage. Such supplements are intended to provide additional nutrients and/or a health benefit to the subject consuming it. A supplement can be used for providing nutrients and/or a health benefit to human beings, as well as to animals, as defined above. Supplements include for example powder supplements to be added to breast milk, for example for premature or low birth weight infants.

In another particular embodiment the microbiota-modulating agent is in the form of a pharmaceutical product. Pharmaceutical products include for example drops, syrups, powder, tablet or capsule products intended to treat of prevent an adverse medical condition in a subject in need thereof.

A nutritional composition of the invention, and especially the infant formula, generally contains a protein source, a carbohydrate source and a lipid source. In some embodiments however, especially if a nutritional composition of the invention is a supplement or a fortifier, there may be only lipids (or a lipid source).

A nutritional composition according to the invention may contain a protein source. The protein may be in an amount of from 1.6 to 3 g per 100 kcal. In some embodiments, especially when the composition is intended for preterm infants/young children, the protein amount can be between 2.4 and 4 g/100 kcal or more than 3.6 g/100 kcal. In some other embodiments, the protein amount can be below 2.0 g per 100 kcal, e.g. between 1.8 to 2 g/100 kcal, or in an amount below 1.8g per 100 kcal.

Protein sources based on, for example, whey, casein and mixtures thereof may be used as well as plant based protein sources, for example, based on soy. As far as whey proteins are concerned, the protein source may be based on acid whey or sweet whey or mixtures thereof and may include alpha-lactalbumin and beta-lactoglobulin in any desired proportions. In some embodiments the protein source is whey predominant (i.e. more than 50% of proteins are coming from whey proteins, such as 60%>or 70%>). The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids.

The proteins may be either fully or partially hydrolysed. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% >by weight of lysine; for example about 7% by weight of lysine which greatly improves the nutritional quality of the protein source.

In one particular embodiment, the proteins of the composition are hydrolysed, fully hydrolysed or partially hydrolysed. The degree of hydrolysis (DH) of the protein can be between 2 and 20, or between 8 and 40, or between 20 and 60 or between 20 and 80 or more than 10, 20, 40, 60, 80 or 90. For example, nutritional compositions containing hydrolysates having a degree of hydrolysis less than about 15% are commercially available from Nestle Company under the trade mark Peptamen®.

At least 70%, 80%, 85%, 90%, 95% or 97% of the proteins may be hydrolysed. In a particular embodiment, 100% of the proteins are hydrolysed.

In one particular embodiment the proteins of the composition are plant based protein.

A nutritional composition according to the present invention may contain a carbohydrate source. This is particularly preferable in the case where a nutritional composition of the invention is an infant formula. In this case, any carbohydrate source conventionally found in infant formulae such as lactose, sucrose, saccharose, maltodextrin, starch and mixtures thereof may be used although one of the preferred sources of carbohydrates for infant formula is lactose.

A nutritional composition according to the present invention may contain lipids and essential fatty acids. Non limiting examples of lipids include: palm olein, high oleic sunflower oil, high oleic safflower oil, canola oil, fish oil, coconut oil, bovine milk fat, and combinations thereof. It may be particularly beneficial if the composition comprises fat in an amount of 25 to 30 g/100 g dry weight of the composition. Non limiting examples of essential fatty acids include: linoleic acid (LA), α-linolenic acid (ALA). Compositions of the invention may further contain gangliosides monosialoganglioside-3 (GM3) and disialogangliosides 3 (GD3), and combinations thereof.

A nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B3, vitamin B6, vitamin B12, vitamin E, vitamin K1, vitamin K2, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population. If necessary, a nutritional composition of the invention may contain emulsifiers and stabilisers such as soy, lecithin, citric acid esters of mono-and diglycerides, and the like.

The composition of the invention may also contain other substances which may have a beneficial effect, especially on bone health or bone development, such as lactoferrin, osteopontin, TGFbeta, slgA, glutamine, nucleotides, nucleosides, and the like.

### Preparation of Compositions

The compositions according to the present invention may be prepared by any known or otherwise suitable manner. For example, a nutritional composition, e.g. an infant formula, may be proposed by blending together a source of protein with a carbohydrate source and a lipid source in appropriate proportions. If used, emulsifiers may be included at this stage. Vitamins and minerals may be added at this stage, but may also be added later to avoid thermal degradation. Water, preferably water which has been subjected to reverse osmosis or deionized water, may then be added and mixed in to form a liquid mixture. The temperature of mixing is preferably room temperature, but may also be higher. The liquid mixture may then be thermally treated to reduce bacterial loads. The mixture may then be homogenized.

If it is desired to produce a powdered composition, the homogenized mixture is dried in a suitable drying apparatus, such as a spray drier or freeze drier and converted into powder.

Processes used in the manufacture of formulae for infants and young children are based on the concept that the products must be nutritionally adequate and microbiologically safe to consume. Thus, steps that eliminate or restrict microbiological growth are central to production processes. The processing technology for each specific formula is proprietary to the manufacturer but, in general, it involves the preservation of an oil-in-water (o/w) emulsion by dehydration in the case of powder products or, sterilization in the case of ready-to-feed or concentrated liquid products. Powdered infant formula may be produced using various processes, such as dry blending dehydrated ingredients to constitute a uniform formula or hydrating and wet-mixing a mixture of macro-ingredients, such as fat, protein and carbohydrate ingredients and then evaporating and spray drying the resultant mixture. A combination of the two processes described above may be used where a base powder is first produced by wet-mixing and spray drying all or some of the macro-ingredients and then dry blending the remaining ingredients, including carbohydrate, minerals and vitamins and other micronutrients, to create a final formula. Liquid formulae are available in a ready-to-feed format or as a concentrated liquid, which requires dilution, normally 1:1, with water. The manufacturing processes used for these products are similar to those used in the manufacture of recombined milk.

If it is desired to produce a liquid infant formula, the homogenized mixture may be filled into suitable containers, preferably aseptically. However, the liquid composition may also be retorted in the container, suitable apparatus for carrying out the filling and retorting of this nature is commercially available.

### Enhancing Bone Development and/or Bone Strength

The present inventors have shown that bone development and/or bone strength in a subject is associated with the abundance of Intestinimonas butyriciproducens, Turicibacter sanguinis, Paraprevotella clara, and Acidaminococcaceae in the gut of the subject. In particular, it the abundances of Intestinimonas butyriciproducens and Turicibacter sanguinis are positively associated with bone outcomes and the abundances of Paraprevotella clara and Acidaminococcaceae are negatively associated with bone outcomes.

The microbiota-modulating agent of the present invention modulates the abundance of one or more bacterial taxa selected from Intestinimonas butyriciproducens, Turicibacter sanguinis, Paraprevotella clara, and Acidaminococcaceae in the gut of a subject and may therefore be used to enhance bone development and/or bone strength in the subject.

In one aspect, the present invention provides a microbiota-modulating agent according to the present invention for use in enhancing bone development and/or bone strength in a subject.

In one aspect, the present invention provides a method for enhancing bone development and/or bone strength in a subject, said method comprising administering a therapeutically effective amount of a microbiota-modulating agent according to the present invention to a subject in need thereof.

Within the context of the present invention, the term “enhancing bone development and/or bone strength” may refer to, in particular, one or more of the following physiological processes: bone catch-up growth, bone mass acquisition, optimization of peak bone mass, promotion of bone formation, promotion of bone anabolism, increase of bone mineral density and micro-architecture, modulation of bone biomechanical properties, modulation the ratio of bone formation and/or bone resorption, assist bone regeneration during fracture healing, regulation of bone resorption process.

The microbiota-modulating agent of the present invention may enhance bone development. As used herein, “promoting bone development” may refer to the support of normal bone metabolism, for example during childhood and adolescence, and/or homeostasis. During childhood and adolescence bones are sculpted by a process called modelling, which allows for the formation of new bone at one site and the removal of old bone from another site within the same bone. Bone remodelling is a lifelong process where mature bone tissue is removed from the skeleton and new bone tissue is formed. Supporting normal bone metabolism and/or homeostasis may refer to support of bone normal modelling and/or remodelling. Supporting normal bone metabolism and/or homeostasis may result in normal bone anatomy and physiology.

The microbiota-modulating agent of the present invention may enhance bone growth and/or strength. As used herein, “promoting bone growth and/or strength” may refer to the support of normal bone growth and/or strength, for example during childhood and adolescence. Supporting normal bone growth and/or strength may result in normal bone anatomy and physiology. Suitable methods and parameters to determine bone growth and bone strength will be known to the skilled person (see e.g. Donnelly, E., 2011. Clinical Orthopaedics and Related Research, 469(8), pp. 2128-2138). Suitably, normal bone growth and/or strength may be determined using one or more bone parameter selected from: trabecular bone volume fraction (BV/TV), bone mineral density (BMD), bone mineral content (BMC), cortical bone volume (Ct.BV), medio-lateral diameter, antero-posterior diameter, bone ultimate force (FMax), and bone stiffness. In some embodiments, normal bone growth and/or strength is determined using one or more bone parameter selected from: bone mineral density (BMD), trabecular bone volume fraction (BV/TV), cortical bone volume (Ct.BV), and bone ultimate force (FMax). Suitable methods to determine these parameters will be available to the skilled person.

The microbiota-modulating agent of the present invention may promote catch-up growth in subjects with stunted growth. Neonatal bone maturation may be a predictive factor of height gain in children born small for gestational age during the first year of life (see e.g. Pepe, G., et al., 2020. Frontiers in Endocrinology, 11, p. 147). Suitable method and parameters to determine catch-up growth will be known to the skilled person. Suitably, catch-up growth may be determined using height velocity.

The microbiota-modulating agent of the present invention may enhance bone healing. As used herein, “promoting bone healing” may refer to the support of normal bone healing, for example following fractures. Fractures are one of the most frequent injuries of the musculoskeletal system. Although fracture treatment has improved considerably in recent decades, a large proportion of all fractures still display delayed healing and complications including non-union. Thus, supporting normal bone healing may, for example, prevent delayed union and/or non-union. Increasing age may increase the risk of delayed union or non-union. The microbiota-modulating agent of the present invention may prevent and/or reduce the frequency and/or occurrence and/or severity and/or duration of fractures.

The microbiota-modulating agent of the present invention may increase the abundance of Intestinimonas butyriciproducens and/or Turicibacter sanguinis in the gut of the subject and/or decrease the abundance of Paraprevotella clara and/or Acidaminococcaceae in the gut of a subject, thereby enhancing bone development and/or bone strength in the subject.

The microbiota-modulating agent of the present invention may increase the abundance of Intestinimonas butyriciproducens and/or Turicibacter sanguinis in the gut of a subject, thereby enhancing bone development and/or bone strength in the subject.

The microbiota-modulating agent of the present invention may increase the abundance of Intestinimonas butyriciproducens in the gut of a subject, thereby enhancing bone development and/or bone strength in the subject. The present inventors have shown that the abundance of Intestinimonas butyriciproducens in the gut of a subject is particularly positively associated with bone microarchitecture, in particular trabecular structure. In some embodiments, the microbiota-modulating agent of the present invention increases the abundance of Intestinimonas butyriciproducens in the gut of the subject, thereby enhancing development of bone microarchitecture, in particular trabecular structure.

The microbiota-modulating agent of the present invention may increase the abundance of Turicibacter sanguinis in the gut of a subject, thereby enhancing bone development and/or bone strength in the subject. The present inventors have shown that the abundance of Turicibacter sanguinis in the gut of a subject is particularly positively associated with bone strength. In some embodiments, the microbiota-modulating agent of the present invention increases the abundance of Turicibacter sanguinis in the gut of the subject, thereby enhancing bone strength.

The microbiota-modulating agent of the present invention may decrease the abundance of Paraprevotella clara and/or Acidaminococcaceae in the gut of a subject, thereby enhancing bone development and/or bone strength in the subject.

The microbiota-modulating agent of the present invention may decrease the abundance of Paraprevotella clara in the gut of a subject, thereby enhancing bone development and/or bone strength in the subject. The present inventors have shown that the abundance of Paraprevotella clara in the gut of a subject is particularly negatively associated with bone microarchitecture. In some embodiments, the microbiota-modulating agent of the present invention decreases the abundance of Paraprevotella clara in the gut of the subject, thereby enhancing development of bone microarchitecture, in particular trabecular structure.

The microbiota-modulating agent of the present invention may decrease the abundance of Acidaminococcaceae in the gut of a subject, thereby enhancing bone development and/or bone strength in the subject. The present inventors have shown that the abundance of Acidaminococcaceae in the gut of a subject is particularly negatively associated bone strength. In some embodiments, the microbiota-modulating agent of the present invention decreases the abundance of Acidaminococcaceae in the gut of the subject, thereby enhancing bone strength.

The “abundance” of a bacterial taxa in the gut of a subject may be determined by any suitable method (see e.g. Tang, Q., et al., 2020. Frontiers in cellular and infection microbiology, 10, p. 151). Suitably, the abundance of bacterial taxa may be obtained from or obtainable from fecal samples. The abundance may be a relative abundance and/or absolute abundance. Preferably, the abundance is a relative abundance, for example, the abundance may be calculated relative to total bacterial abundance in the gut of the subject.

### Subject and Route of Administration

The subject may be any suitable subject. Suitably, the subject may be a mammal. In preferred embodiments, the subject is a human. In other embodiments, the subject is an animal, preferably wherein the animal is a pet. A pet may be an animal selected from dogs, cats, birds, fish, rodents such as mice, rats, and guinea pigs, rabbits, etc.

The present invention is particularly suitable for infants and young children at risk of bone disease, having a family history of bone disease, or having already experienced at least one, preferably several, episode(s) of fracture. The present invention is also particularly suitable for infants and young children who were born preterm or with low-birth weight or experienced intra-uterine growth retardation or who suffered from growth stunting because of malnutrition or experienced disease such as Crohn's disease and/or celiac disease and/or cancer or who were treated with drugs leading to malabsorption, anorexia and/or metabolic bone disease, such as chemotherapy drugs and/or corticosteroids. The present invention is particularly preferred for use in infants and children who were born preterm or with low-birth weight or experienced intra-uterine growth retardation, or with intra-uterine malnutrition or who suffered growth delay.

In some embodiments, the subject is a juvenile, an adolescent, a child, or an infant. The term “juvenile” may refer to an individual that has not yet reached adulthood. The term “adolescent” may refer to an individual during the period from the onset of puberty to adulthood. The term “child” may refer an individual between the stages of birth and puberty.

In some embodiments, the subject is an infant, a toddler, or a young child. The term “infant” may refer to a subject aged from about 0 years to about 1 year. The term “toddler” may refer to a subject aged from about 1 year to about 3 years. The term “young child” may refer to a subject aged from about 3 years to about 5 years.

In some embodiments, the subject was born preterm or with low-birth weight or experienced intra-uterine growth retardation. The term “preterm infant” may refer to an infant born at least than 37 weeks gestational age. The term “low birth weight infant” may refer to an infant having a live-born weight less than 2,500 g.

In some embodiments, the subject suffered from and/or is suffering from stunted growth. The definition of stunting may refer to the “height for age” value to be less than two standard deviations of the WHO Child Growth Standards median (see e.g. De Onis, M. and Branca, F., 2016. Maternal & child nutrition, 12, pp. 12-26). In some embodiments, the subject suffered from and/or is suffering from faltering growth. The term “faltering growth” may describe a pattern of slower weight gain than expected for age and sex in infants, children and other adolescents (see e.g. King, C. and Davis, T., 2010. European journal of clinical nutrition, 64(1), pp. S11-S13). In some embodiments, the subject suffered from and/or is suffering from growth stunting and/or faltering growth because of malnutrition or experienced disease such as anorexia, Crohn's disease and/or celiac disease. In some embodiments, the subject suffered from and/or is suffering from growth stunting and/or faltering growth because of treatment with drugs leading to malabsorption, anorexia and/or metabolic bone disease, such as chemotherapy drugs and/or corticosteroids.

The present invention can also apply to adolescents or adults at risk of bone disease or having experienced at least one, preferably several, episode(s) of fractures, or who were born preterm or with low-birth weight or experienced intra-uterine growth retardation of who suffered from growth stunting because of malnutrition or experienced disease such as Crohn's disease and/or celiac disease and/or cancer or who were treated with drugs leading to malabsorption, anorexia and/or metabolic bone disease, such as chemotherapy drugs and/or corticosteroids or who suffered from growth delays because of disease or malnutrition or drugs' use during infancy and/or childhood (including adolescence).

In some embodiments, the subject is an adolescent or an adult. In some embodiments, the subject is an adult, preferably wherein the subject is elderly. In some embodiments, the subject is at least 60 years of age, at least 65 years of age, at least 70 years of age, at least 75 years of age, or at least 80 years of age. The subject may have one or more fractures. The subject may have or may be at risk of one or more delayed union and/or one or more non-union.

The microbiota-modulating agent may be administered by any suitable method known to the skilled person. For example, the microbiota-modulating agent may be administered by oral and/or enteral administration. In some embodiments, the microbiota-modulating agent is orally administered.

### EXAMPLES

The invention is further described with reference to the following examples. It will be appreciated that the invention as claimed is not intended to be limited in any way by these examples.

### Example 1

Female Göttingen Minipigs (n=48, Ellegaard, Denmark) were randomly allocated to be artificially reared with milk substitutes containing blends of either sialylated (2-HMOs: 3′SL and 6′SL; 0.68 g/L, hereafter referred to as “HMO blend A”), neutral (4-HMOs: LNnT, LNT, 2′FL and di-FL; 4 g/L, hereafter referred to as “HMO blendB”), both sialylated or neutral (6 HMOs: 4 g/L, hereafter referred to as “HMO blend C”) human milk oligosaccharides (HMOs) or lactose (C: 4 g/L, hereafter referred to as “M”) from 10 days to 11 weeks of age (weaning). Starting at weaning piglets were offered equal controlled amounts of a mild-western chow diet (13% protein, 14% fat, 33% cho, 4.4 MCals GE/kg) up to 48 weeks of age with ad libitum water. A naturally-reared reference group of piglets (NR; n=12) was kept with the sow until weaning and was fed the same diet afterwards. The minipigs were sacrificed at 12 months of age (adulthood equivalence). Tibia was directly excised and stored at −20C. Feces were collected at same time point and stored at −20C.

### 1) Measurement of Bone Mineral Density (BMD)

BMD was measured by Dual energy X-ray absorptiometry (or DXA). BMD corresponds to the bone mineral content divided by the surface of the tissue analysed and it is express in g/cm². Minipigs bones are in the range of human children bones sizes. We therefore use a DXA scanner with human parameters to evaluate the BMD of minipig tibias. Equipment used: Lunar iDXA (GE Healthcare) localized at VCLB metabolic unit and software: EnCORE 2011 (version 13.60.003, GE Healthcare). BMD have been analysed for the all tibia and different region of interest (proximal, midshaft and distal tibia) previously described in the literature (Bonnet N, et al. Bone. 2005; 37 (5): 622-33).

### 2) Analysis of Bone Trabecular and Cortical Microstructure

Micro-computed tomography (μCT UCT40, Scanco Medical AG, Basserdorf Switzerland) was used to assess trabecular and cortical microstructure respectively investigated at proximal metaphysis and midshaft diaphysis tibia as previously described (Bonnet N, et al. J Bone Miner Res. 2017; doi: 10:1002). Briefly, trabecular and cortical bone regions were evaluated using isotropic 12 μm voxels.

For the tibial trabecular region, to eliminate the primary spongiosa, 200 slices taken from the 100 slices under the proximal growth plate were analysed. Tibial cortical structure was assessed using 50 continuous CT slides (600 μm) located at the tibial midshaft. Morphometric variables were computed from binarized images using direct, three-dimensional techniques that do not rely on prior assumptions about the underlying structure (Bonnet N, et al. Med Phys 2009; 36 (4): 1286-97).

For the trabecular bone regions, the bone volume fraction (BV/TV) was assessed. For cortical bone at the femoral and tibial midshaft, the Cortical Bone Volume (Ct.BV, mm³) was measured.

### 3) Analysis of Bone Biomechanics

In order to test the biomechanical properties of the bone an axial compression test was performed on tibias. The load was applied in a compression mode at a nominal deformation rate of 2 mm/minute until fracture. Load-displacement curves were recorded during testing. Ultimate load and failure energy were determined.

### 4) Microbiome Analysis

Analyses of microbiome composition (taxa abundances) from the fecal samples was done and statistical correlation with bone outcomes were performed using Spearman correlation to highlight association between taxa abundance and bone phenotypes. The pig/minipig microbiota gene catalog containing 6,601,931 genes was used to identify bacterial taxa. The library was sequenced using 2×150 bp paired-end sequencing on an Illumina platform. The approach is based on the metagenomic species concept.

### 5) Results

Analysis by bacterial genus showed that Intestinimonas, Turicibacter and Paraprevotella were among the top 10 bacterial genus associated with different bone outcomes. In particular, the Turicibacter genus was positively associated with tibial bone mineral density, cortical structure, and bone strength and the Intestinimonas genus and Paraprevotella genus were associated with trabecular structure. The results are shown in the table below.

| | | | | | |
|---|---|---|---|---|---|
| Genus | Estimate | StdError | t-value | p-value | fdr |

Tibial bone mineral density

| | | | | | |
|---|---|---|---|---|---|
| Turicibacter | 0.12 | 0.05 | 2.55 | 0.0135 | 0.2365 |
| Paraprevotella | −0.95 | 0.60 | −1.59 | 0.1170 | 0.4766 |

Trabecular structure

| | | | | | |
|---|---|---|---|---|---|
| Paraprevotella | −788.30 | 207.53 | −3.80 | 0.0004 | 0.0096 |
| Intestimonas | 360.48 | 101.41 | 3.55 | 0.0008 | 0.0096 |
| Turicibacter | 49.20 | 17.30 | 2.84 | 0.0063 | 0.0314 |

Cortical structure

| | | | | | |
|---|---|---|---|---|---|
| Turicibacter | 95.95 | 32.45 | 2.96 | 0.0048 | 0.1504 |

Bone strength

| | | | | | |
|---|---|---|---|---|---|
| Turicibacter | 2.19 | 0.70 | 3.12 | 0.0032 | 0.1105 |
| Paraprevotella | −13.42 | 9.24 | −1.45 | 0.1533 | 0.7664 |

Analysis by bacterial taxa showed that Intestinimonas butyriciproducens, Turicibacter sanguinis, Paraprevotella clara, and Acidaminococcaceae were each among the top 10 bacterial taxa associated with different bone outcomes. In particular: (i) Intestinimonas butyriciproducens was positively associated with trabecular structure; (ii) Turicibacter sanguinis was positively associated with bone strength; (iii) Paraprevotella clara was negatively associated with trabecular structure; and (iv) Acidaminococcaceae was negatively correlated with bone strength. The results are shown in the table below.

| | | | |
|---|---|---|---|
| Taxa | Estimate | p-value | fdr |

Trabecular structure

| | | | |
|---|---|---|---|
| Paraprevotella clara | −0.54 | 0.00002 | 0.01 |
| Intestimonas butyriciproducens | 0.47 | 0.00033 | 0.08 |

Bone strength

| | | | |
|---|---|---|---|
| Acidaminococcaceae | −0.38 | 0.00852 | 0.56 |
| Turicibacter sanguinis | 0.37 | 0.01032 | 0.56 |

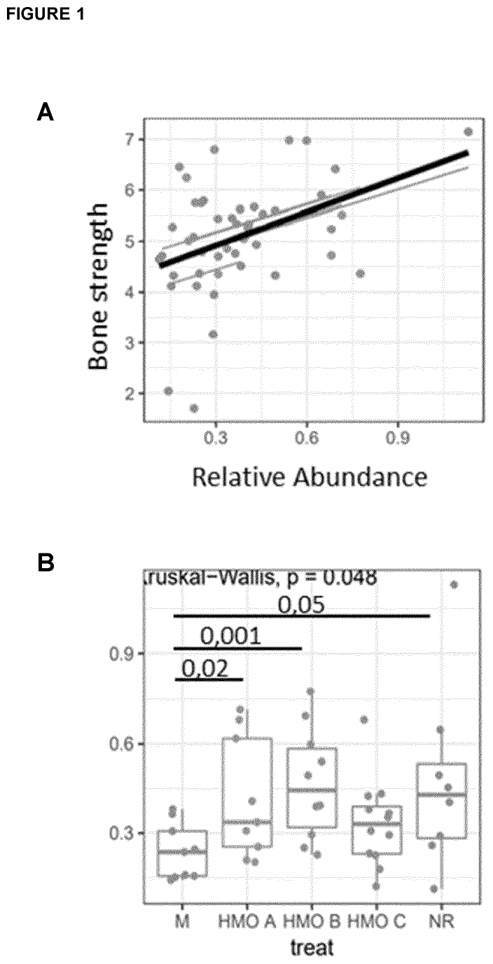

Further analysis confirmed that Turicibacter sanguinis is positively associated with bone strength (r=0.37, p<0.01) (see FIG. 1A). Turicibacter sanguinis is also positively correlated with BMD (r=0.26, p<0.05), trabecular structure (r=0.28, p<0.05), and cortical structure (r=0.36, p<0.01). The relative abundance of Turicibacter sanguinis in minipigs reared with milk substitutes was increased by each of HMO blends A, B, and C and reached similar levels compared to naturally-reared subjects (see FIG. 1B).

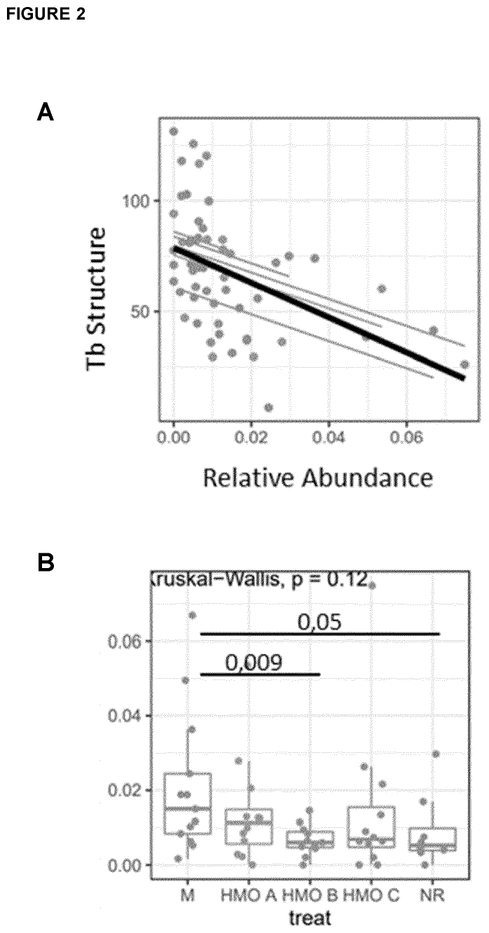

Further analysis confirmed that Paraprevotella clara is negatively correlated with trabecular structure (r=−0.54, p<0.0001) (see FIG. 2A). Paraprevotella clara is also negatively correlated with BMD (r=0.31, p<0.05) and bone strength (r=−0.29, p<0.05). The relative abundance of Paraprevotella clara in minipigs reared with milk substitutes was decreased by each of HMO blends A, B, and C and reached similar levels compared to naturally-reared subjects (see FIG. 2B).

### Embodiments

Various preferred features and embodiments of the present invention will now be described with reference to the following numbered paragraphs (paras).
1. A microbiota-modulating agent for use in enhancing bone development and/or bone strength in a subject, wherein the microbiota-modulating agent modulates the abundance of one or more bacterial taxa selected from Intestinimonas butyriciproducens, Turicibacter sanguinis, Paraprevotella clara, and Acidaminococcaceae in the gut of the subject.
2. A method for enhancing bone development and/or bone strength in a subject, said method comprising administering a therapeutically effective amount of a microbiota-modulating agent to a subject in need thereof, wherein the microbiota-modulating agent modulates the abundance of one or more bacterial taxa selected from Intestinimonas butyriciproducens, Turicibacter sanguinis, Paraprevotella clara, and Acidaminococcaceae in the gut of the subject.
3. The microbiota-modulating agent for use according to para 1 or method according to para 2, wherein the microbiota-modulating agent is Intestinimonas butyriciproducens and/or Turicibacter sanguinis.
4. The microbiota-modulating agent for use according to para 1 or method according to para 2, wherein the microbiota-modulating agent is a mixture of oligosaccharides comprising at least one sialylated oligosaccharide, at least one fucosylated oligosaccharide and/or at least one N-acetylated oligosaccharide.
5. The microbiota-modulating agent for use according to para 1 or method according to para 2, wherein the microbiota-modulating agent is a combination comprising a mixture of oligosaccharides and Intestinimonas butyriciproducens and/or Turicibacter sanguinis, wherein said mixture of oligosaccharides comprises at least one sialylated oligosaccharide, at least one fucosylated oligosaccharide and/or at least one N-acetylated oligosaccharide, and wherein the mixture of oligosaccharides and Intestinimonas butyriciproducens and/or Turicibacter sanguinis are administered separately, simultaneously or sequentially.
6. The microbiota-modulating agent for use or method according to para 3 or 5, wherein the Intestinimonas butyriciproducens and/or Turicibacter sanguinis is administered in a dose of 1E6 to 1E10 cfu/day.
7. The microbiota-modulating agent for use or method according to any of paras 4 to 6, wherein the at least one sialylated oligosaccharide is selected from the group consisting of 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof.
8. The microbiota-modulating agent for use or method according to any one of paras 4 to 7, wherein the at least one sialylated oligosaccharide is selected from 3′-sialyllactose (3′-SL), 6′-sialyllactose (6′-SL) and combinations thereof.
9. The microbiota-modulating agent for use or method according to any one of paras 4 to 8, wherein the mixture comprises 3′-sialyllactose (3′-SL) and 6′-sialyllactose (6′-SL).
10. The microbiota-modulating agent for use or method according to any one of paras 4 to 9, wherein the at least one fucosylated oligosaccharide is selected from the group consisting of 2′-fucosyllactose (2′FL), 3-fucosyllactose (3FL), difucosyllactose (diFL), lacto-N-fucopentaose-I (LNFP-I), lacto-N-fucopentaose-II (LNFP-II), lacto-N-fucopentaose-III (LNFP-III), lacto-N-fucopentaose-V (LNFP-V), lacto-neofucopentaose V (LNnFP-V), lacto-N-difucosylhexaose-I (LNDFH-1), lacto-N-neodifucosylhexaose (LNnDFH), monofucosyllacto-n-hexaose-III (MFNLH-III), difucosyllacto-N-hexaose-a (DFLNHa) and combinations thereof.
11. The microbiota-modulating agent for use or method according to any one of paras 4 to 10, wherein the at least one fucosylated oligosaccharide is selected from the group consisting of 2′-fucosyllactose (2′FL), difucosyllactose (diFL), and combinations thereof.
12. The microbiota-modulating agent for use or method according to any one of paras 4 to 11, wherein the mixture comprises 2′-fucosyllactose (2′FL) and difucosyllactose (diFL).
13. The microbiota-modulating agent for use or method according to any one of paras 4 to 12, wherein the at least one N-acetylated oligosaccharide is selected from the group consisting of N-acetyl-glucosamine, N-acetyl-galactosamines, lacto-N-tetraose (LNT), lacto-N-neotetraose (LNnT), and combinations thereof.
14. The microbiota-modulating agent for use or method according to any one of paras 4 to 13, wherein said at least one N-acetylated oligosaccharide is selected from lacto-N-tetraose (LNT), lacto-N-neotetraose (LNnT) and combinations thereof.
15. The microbiota-modulating agent for use or method according to any one of paras 4 to 14, wherein the mixture comprises lacto-N-tetraose (LNT) and lacto-N-neotetraose (LNnT).
16. The microbiota-modulating agent for use or method according to any one of paras 4 to 15, wherein the mixture comprises or consists of 2′-fucosyllactose (2′FL), difucosyllactose (diFL), lacto-N-tetraose (LNT) and lacto-N-neotetraose (LNnT), optionally wherein the mixture comprises or consists of 3′-sialyllactose (3′-SL), 6′-sialyllactose (6′-SL), 2′-fucosyllactose (2′FL), difucosyllactose (diFL), lacto-N-tetraose (LNT) and lacto-N-neotetraose (LNnT).
17. The microbiota-modulating agent for use or method according to any one of paras 4 to 16, wherein the oligosaccharide mixture comprises:
   −10 to 35 wt %, preferably 10 to 30 wt %, more preferably 10-25 wt %, with respect to the total weight of the oligosaccharide mixture, of at least one sialylated oligosaccharide;
   −30 to 80 wt %, preferably 40 to 80 wt %, more preferably 50 to70 wt %, with respect to the total weight of the oligosaccharide mixture, of at least one fucosylated oligosaccharide; and/or
   −10 to 35 wt %, preferably 15 to 30 wt %, more preferably 15 to 20 wt %, with respect to the total weight of the oligosaccharide mixture, of at least one N-acetylated oligosaccharide.
18. The microbiota-modulating agent for use or method according to any preceding para, wherein the microbiota-modulating agent is in the form of a nutritional composition, a medical food product for clinical nutrition, or a supplement.
19. The microbiota-modulating agent for use or method according to any preceding para, wherein the microbiota-modulating agent is in the form of an infant formula.
20. The microbiota-modulating agent for use or method according to any preceding para, wherein the microbiota-modulating agent is in the form of a preterm infant formula, a human milk fortifier, a starter infant formula, a follow-on formula, a baby-food formula, an infant cereal formula, or a growing-up milk.
21. The microbiota-modulating agent for use or method according to any one of paras 18 to 20, wherein the composition comprises Intestinimonas butyriciproducens and/or Turicibacter sanguinis in an amount of 1E5 to 1E9 cfu/g.
22. The microbiota-modulating agent for use or method according to any one of paras 18 to 21, wherein the composition comprises:
   −0.01 to 2 wt %, preferably 0.05 to 1.5wt %, most preferably 0.07% to 1 wt %, with respect to the total weight of the composition in powder form, of at least one sialylated oligosaccharide;
   −0.05 to 3 wt %, preferably 0.1 to 2 wt %, most preferably 0.2 to 1.5 wt %, with respect to the total weight of the composition in powder form, of at least one fucosylated oligosaccharide; and/or
   −0.01 to 1 wt %, preferably 0.03 to 0.6 wt %, most preferably 0.05 to 0.5 wt %, with respect to the total weight of the composition in powder form, of at least one N-acetylated oligosaccharide.
23. The microbiota-modulating agent for use or method according to any one of the preceding paras, wherein the subject is a juvenile, an adolescent, a child, a toddler, or an infant, optionally wherein the is suffering from stunted and/or faltering growth.
24. The microbiota-modulating agent for use or method according to any one of paras 1 to 22, wherein the subject is an adult, optionally wherein the subject is elderly and/or at risk of delayed union and/or non-union.
25. The microbiota-modulating agent for use or method according to any one of the preceding paras, wherein the subject is a human or an animal, preferably wherein the subject is a human.
26. The microbiota-modulating agent for use or method according to any one of the preceding paras, wherein the microbiota-modulating agent increases the abundance of Intestinimonas butyriciproducens and/or Turicibacter sanguinis in the gut of the subject and/or the microbiota-modulating agent decreases the abundance of Paraprevotella clara and/or Acidaminococcaceae in the gut of the subject.
27. The microbiota-modulating agent for use or method according to any one of the preceding paras, wherein the enhancement of bone development and/or bone strength comprises at least one of the following physiological processes: bone catch-up growth, bone mass acquisition, optimization of peak bone mass, promotion of bone formation, promotion of bone anabolism, increase of bone mineral density and micro-architecture, modulation of bone biomechanical properties, modulation the ratio of bone formation and/or bone resorption, assist bone regeneration during fracture healing, regulation of bone resorption process.
28. The microbiota-modulating agent for use or method according to any one of the preceding paras, wherein the microbiota-modulating agent increases one or more bone parameter selected from: bone mineral density (BMD), trabecular bone volume fraction (BV/TV), cortical bone volume (Ct.BV) and bone ultimate force (FMax).
29. Use of a microbiota-modulating agent for modulating the abundance of one or more bacterial taxa selected from Intestinimonas butyriciproducens, Turicibacter sanguinis, Paraprevotella clara, and Acidaminococcaceae in the gut of a subject.
30. The use according to para 29, wherein the microbiota-modulating agent is defined according to any one of paras 3 to 26.
31. A microbiota-modulating agent comprising Intestinimonas butyriciproducens and/or Turicibacter sanguinis.
32. The microbiota-modulating agent according to para 31, wherein the microbiota-modulating agent is a probiotic or a synbiotic.
33. The microbiota-modulating agent according to para 31 or 32, wherein the microbiota-modulating agent is defined according to any one of paras 5 to 22.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

1. A method for enhancing bone development and/or bone strength in a subject in need thereof, wherein the microbiota-modulating agent modulates the abundance of one or more bacterial taxa selected from Intestinimonas butyriciproducens, Turicibacter sanguinis, Paraprevotella clara, and Acidaminococcaceae in the gut of the subject comprising administering to the subject a microbiota-modulating agent.

2. The method according to claim 1, wherein the microbiota-modulating agent is Intestinimonas butyriciproducens and Turicibacter sanguinis.

3. The method according to claim 1, wherein the microbiota-modulating agent is an oligosaccharide mixture comprising at least one sialylated oligosaccharide, at least one fucosylated oligosaccharide, and at least one N-acetylated oligosaccharide.

4. The method according to claim 1, wherein the microbiota-modulating agent is a combination comprising an oligosaccharide mixture and Intestinimonas butyriciproducens and/or Turicibacter sanguinis, wherein said oligosaccharide mixture comprises at least one sialylated oligosaccharide, at least one fucosylated oligosaccharide and/or at least one N-acetylated oligosaccharide, and wherein the oligosaccharide mixture and Intestinimonas butyriciproducens and/or Turicibacter sanguinis are administered separately, simultaneously or sequentially.

5. The method according to claim 3, wherein:
(a) the at least one sialylated oligosaccharide is selected from the group consisting of 3′-sialyllactose (3′-SL), 6′-sialyllactose (6′-SL), syalyllacto-N-tetraose b (LSTb), syalyllacto-N-tetraose c (LSTc), disyallacto-N-tetraose, and combinations thereof, preferably wherein the at least one sialylated oligosaccharide is selected from 3′-sialyllactose (3′-SL), 6′-sialyllactose (6′-SL) and combinations thereof;
(b) the at least one fucosylated oligosaccharide is selected from the group consisting of 2′-fucosyllactose (2′FL), 3-fucosyllactose (3FL), difucosyllactose (diFL), lacto-N-fucopentaose-I (LNFP-I), lacto-N-fucopentaose-II (LNFP-II), lacto-N-fucopentaose-III (LNFP-III), lacto-N-fucopentaose-V (LNFP-V), lacto-neofucopentaose V (LNnFP-V), lacto-N-difucosylhexaose-I (LNDFH-1), lacto-N-neodifucosylhexaose (LNnDFH), monofucosyllacto-n-hexaose-III (MFNLH-III), difucosyllacto-N-hexaose-a (DFLNHa) and combinations thereof, preferably wherein the at least one fucosylated oligosaccharide is selected from the group consisting of 2′-fucosyllactose (2′FL), difucosyllactose (diFL), and combinations thereof; and/or
(c) the at least one N-acetylated oligosaccharide is selected from the group consisting of N-acetyl-glucosamine, N-acetyl-galactosamines, lacto-N-tetraose (LNT), lacto-N-neotetraose (LNnT), and combinations thereof, preferably wherein said at least one N-acetylated oligosaccharide is selected from lacto-N-tetraose (LNT), lacto-N-neotetraose (LNnT) and combinations thereof.

6. The method according to claim 3, wherein the oligosaccharide mixture comprises: (a) sialyllactose (3′-SL) and 6′-sialyllactose (6′-SL); (b) 2′-fucosyllactose (2′FL) and difucosyllactose (diFL); and (c) lacto-N-tetraose (LNT) and lacto-N-neotetraose (LNnT).

7. The method according to claim 3, wherein the oligosaccharide mixture comprises:
(a) 10 to 35 wt %, with respect to the total weight of the oligosaccharide mixture, of at least one sialylated oligosaccharide;
(b) 30 to 80 wt %, with respect to the total weight of the oligosaccharide mixture, of at least one fucosylated oligosaccharide; and (c) 10 to 35 wt %, with respect to the total weight of the oligosaccharide mixture, of at least one N-acetylated oligosaccharide.

8. The method according to claim 1, wherein the microbiota-modulating agent is in a form selected from the group consisting of a nutritional composition, a medical food product for clinical nutrition, or a supplement.

9. The method according to claim 1, wherein the microbiota-modulating agent is in the form of an infant formula.

10. The method according to claim 8, wherein the composition comprises:
(a) 0.01 to 2 wt %, of at least one sialylated oligosaccharide, with respect to the total weight of the composition in powder form;
(b) 0.05 to 3 wt %, of at least one fucosylated oligosaccharide, with respect to the total weight of the composition in powder form; and
(c) 0.01 to 1 wt %, of at least one N-acetylated oligosaccharide, with respect to the total weight of the composition in powder form.

11. The method according to claim 1, wherein the subject is a human.

12. The method according to claim 11, wherein the subject is selected from the group consisting of a child, a toddler, and an infant.

13. The method according to claim 1, wherein the microbiota-modulating agent increases the abundance of Intestinimonas butyriciproducens and/or Turicibacter sanguinis in the gut of the subject.

14. The method according to claim 1, wherein the microbiota-modulating agent increases one or more bone parameter selected from the group consisting of bone mineral density (BMD), trabecular bone volume fraction (BV/TV), cortical bone volume (Ct.BV) and bone ultimate force (FMax).

15. A microbiota-modulating agent comprising Intestinimonas butyriciproducens and Turicibacter sanguinis.
